(19) United States
(12) Patent Application Publication
(10) Pub. No.: US 2006/0257501 A1
(43) Pub. Date: Nov. 16, 2006

(54) Composition and method for the therapeutic modulation of matrix metalloproteinase

(76) Inventors: Stephen H. Monroe, (US); Hans Hoekstra, (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/492,569

(22) Filed: Jul. 24, 2006

Related U.S. Application Data
(63) Continuation of application No. 10/645,410, filed on Aug. 21, 2003.
Application No. 10/645,410 is a continuation-in-part of application No. 10/305,713, filed on Nov. 27, 2002, now abandoned.
(60) Provisional application No. 60/334,337, filed on Nov. 29, 2001.

(51) Int. Cl. A61K 31/19; A61K 33/32; A61K 33/00

(52) U.S. Cl. 424/641; 514/574; 424/722

(57) ABSTRACT

A synthesized composition containing one or more of zinc ions, calcium ions, rubidium ions and/or potassium ions in a pharmaceutically acceptable carrier, which, when administered to a patient in need thereof, effectively modulates the activity of at least MMP-2 and/or MMP-9 in the wound. A method is disclosed.

### RELATED APPLICATIONS

This application is a continuation-in-part of copending non-provisional application Ser. No. 10/305,713 filed Nov. 27, 2002, claiming priority based on Provisional application Ser. No. 60/334,337, filed Nov. 29, 2001.

### FIELD OF INVENTION

This invention relates to the use of inorganics as an aid in the establishment and/or control over the chemical environment associated with extra cellular matrices.

More particularly, this application relates to therapeutic modulation of matrix metalloproteinases (MMPs).

In the prior art it is known that there exist within the human body a plurality of matrix metalloproteinases. It has been suggested that at least certain of these MMPs lie relatively dormant (“Pre-MMP”) until activated, whereupon various of the MMPs affect cellular growth or lack of growth, the MMPs acting at least in part through the extracellular matrix (ECM) of the cells

MMP-2 has been particularly indicated in the healing of wounds. In its inactive state. Pro-MMP-2 includes a ribbon of protein which covers its active site. Removal (cleavage) of this protein must occur before this MMP can become activated. This has been termed a “Cysteine switch”. Zinc ions at the active site have been noted to activate MMP-2. Also, calcium ions at a secondary site are believed to provide the MMP with the proper geometry in its active state. Inhibitors of metalloproteinase (TIMP) have been identified.

### SUMMARY OF THE INVENTION

The present inventors have identified MMP-2 and MMP-9 in increased quantities in certain medical conditions. In one such medical condition, MMPs have been noted to be involved both in the peripheral region and particularly within the deep recesses of a chronic wound. It has also been a noted increase in these MMPs in “difficult to heal” open wounds. Further the present inventors have discovered a synthesized composition which, when clinically introduced to a site exhibiting the presence of one or more MMPs effectively shuts down the activity of MMP(s). This therapeutic effect is particularly evident with respect to the modulation of MMP-2 and MMP-9, as evidenced by analysis of wound cultures for the presence of MMPs 2 and 9, and resulting visually observable improvement in the healing of the wound. The visually observable improvement in the healing process of the wound is dramatic and takes place within an unexpectedly short time frame.

Moreover, continued clinical application of the composition of the present invention to a site which exhibits increased or excess MMP values has been found effective in bringing about modulation of such MMPs, with resultant complete recovery of the medical malady which involves the increased or excess MMP values. Such recovery has been noted to take place within unexpectedly short time periods. The composition containing the effective ingredients of the present invention has been determined to be effective in modulating the presence, hence the activity of, MMPs within the deeper inner recesses of wounds and is believed to be effective within other similar or related medical conditions, particularly subsurface traumatized tissue. In clinical environments, wounds such as decubitus ulcers, and deep burns have been effectively treated employing the concepts of the present invention.

### BRIEF DESCRIPTION OF FIGURES

FIG. 1 is a photograph of depicting a wound having applied thereto a composition embodying the present invention;

FIGS. 2-5 are photographs of typical non-responding wounds;

FIGS. 6 and 7 are photographs of the leg wound of Example 1, depicting the wound of Example 1 before and after treatment, respectively, in accordance with the present invention;

FIG. 8 is a photograph of the leg wound of Example 1 before treatment in accordance with the present invention;

FIG. 9 is a microphotograph of a biopsy of the wound depicted in FIG. 8;

FIG. 10 is a microphotograph depicting the levels of MMP-2 in the upper layers of Zones A and B of FIG. 9;

FIG. 11 is a microphotograph depicting the levels of MMP-2 in the deeper layers of Zone C of FIG. 9;

FIG. 12 depicts the appearance of Zones A, B and C of FIG. 9 after 14 days of treatment in accordance with the present invention;

FIG. 13 is a photograph depicting an external view of the wound depicted in FIG. 8 after 14 days of treatment;

FIG. 14 is a microphotograph of Zone B of FIG. 9 after 14 days of treatment;

FIG. 15 is a photograph of the wound of Example 1 after 6 weeks of treatment;

FIG. 16 is a microphotograph of a biopsy of the wound depicted in FIG. 15;

FIG. 17 is a pictorial representation of the wound healing process;

FIG. 18 is a pictorial representation of the balancing of MMPs within a wound;

FIG. 19 is a pictorial representation of ECM generation and degradation in a wound; and,

FIG. 20 is a pictorial representation of collagen formation in a wound.

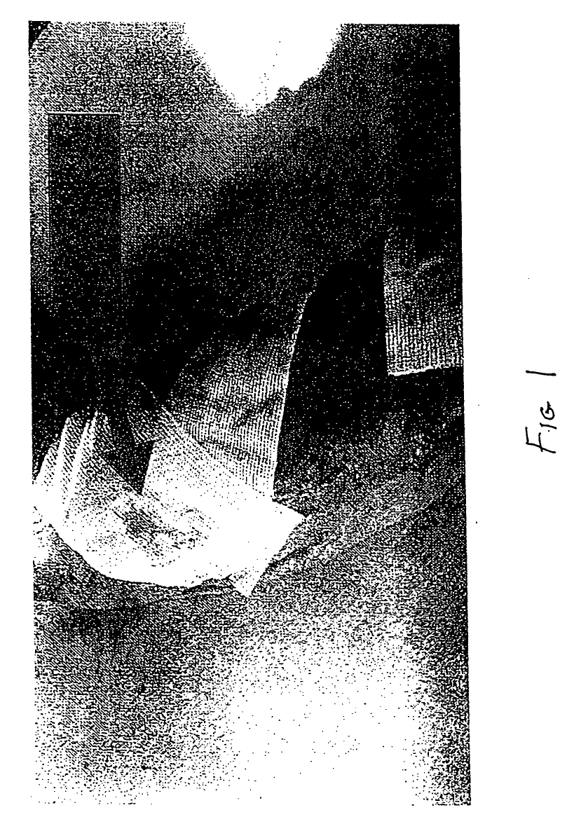

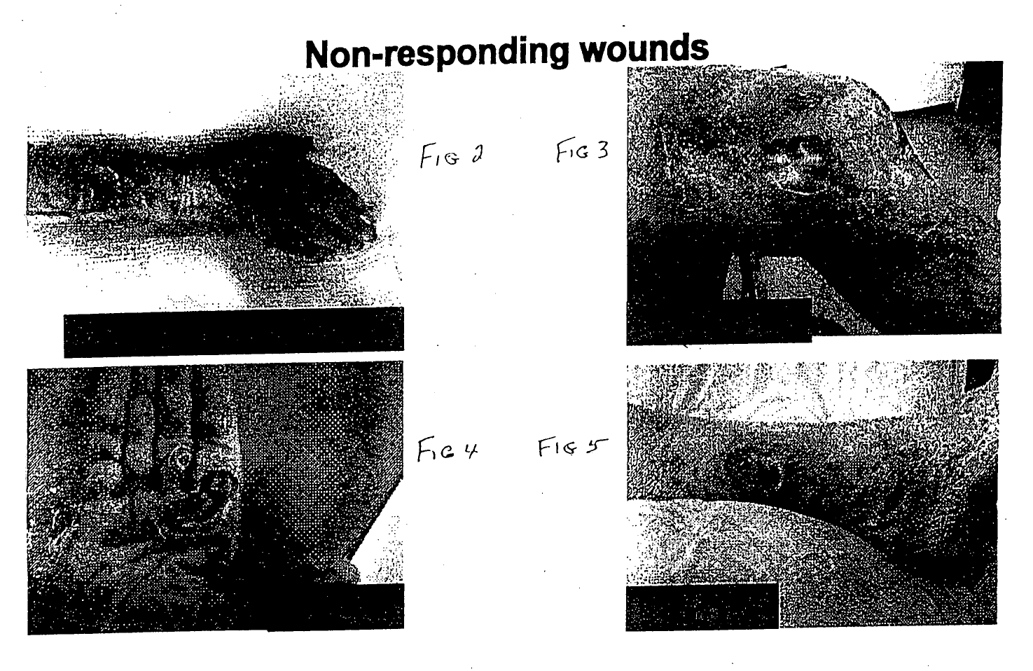

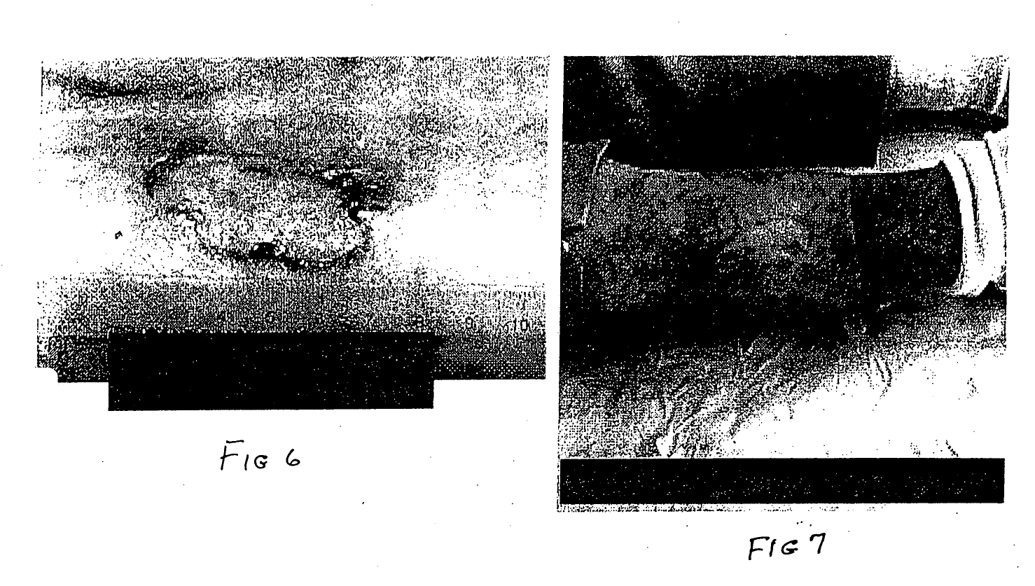

### DETAILED DESCRIPTION OF THE INVENTION

In initial experimentation conducted with rats (partial thickness excision wounds) and Yorkshire pigs (contact burn wounds), the present inventors found that compositions containing the ingredients of the present invention promoted epithelialization, resulting in a more “normal” epidermis. The wound bed contained less activated macrophages, cells staining positive for acid phosphatase.

Infliction of deep dermal contact wounds in domestic pig models induce defects which are not fully epithelialized, depending on the treatment applied. Tissue biopsy wounds are deep full thickness skin defects measuring 9 by 2 cm. Such biopsy wounds have a slow tendency to epithelialize. When excision biopsy wounds are filled up with granulation tissue there is a clear visible healing of the wound by contraction. These wounds are ideal test models to get a clear macroscopic impression of the efficacy of test substances applied. Compositions containing the ingredients of the present invention have been found to convert such wounds, which mainly healed by epithelialization starting a couple of days after the first application. Also, such biopsy wounds showed clear epithelialization instead of contraction in comparison with wounds treated with the present compositions.

Employing-the domestic pig model, compositions containing the ingredients of the present invention were compounded and tested. These tests showed clear expression of MMP-2 in untreated wounds. Only minimal expression of MMP-2 was observed in comparative wounds treated with a composition containing the ingredients of the present invention.

The foregoing tests were followed by in vitro human studies employing a composition containing the ingredients of the present invention. In these tests, the composition was impregnated onto an ethylene vinylacetate carrier to form an impregnated dressing for the wound site.

In the present studies, 31 patients were initially involved in the study. Five patients dropped out of the study and eight patients are receiving continuing treatment. Of these patients, the wound(s) of 18 patients were completely healed with an average healing time of 10 weeks. All of the patients in the study responded positively.

The following specific example is provided as exemplary of the results observed in the human studies. In each patient studied, a composition in accordance with the present invention, on an EVOH carrier defining a bandage was applied to the wound site. The bandage was removed at various intervals and replaced with a fresh bandage. A sufficient quantity of the composition of the present invention was placed on the carrier to substantially fully fill the wound cavity.

### Example I

Female 74 years of age

History:
Rheumatoid Arthritis.

Medication:
High doses of steroids.

Type wound:
Post traumatic ulcer on lateral lower leg after infected hematoma.

Duration of Wound

Wound had existed for more than one year prior to commencement of present treatment.

Earlier Treatments
DUODERM
HYDROGEL
Vacuum system
Honey and SSD,

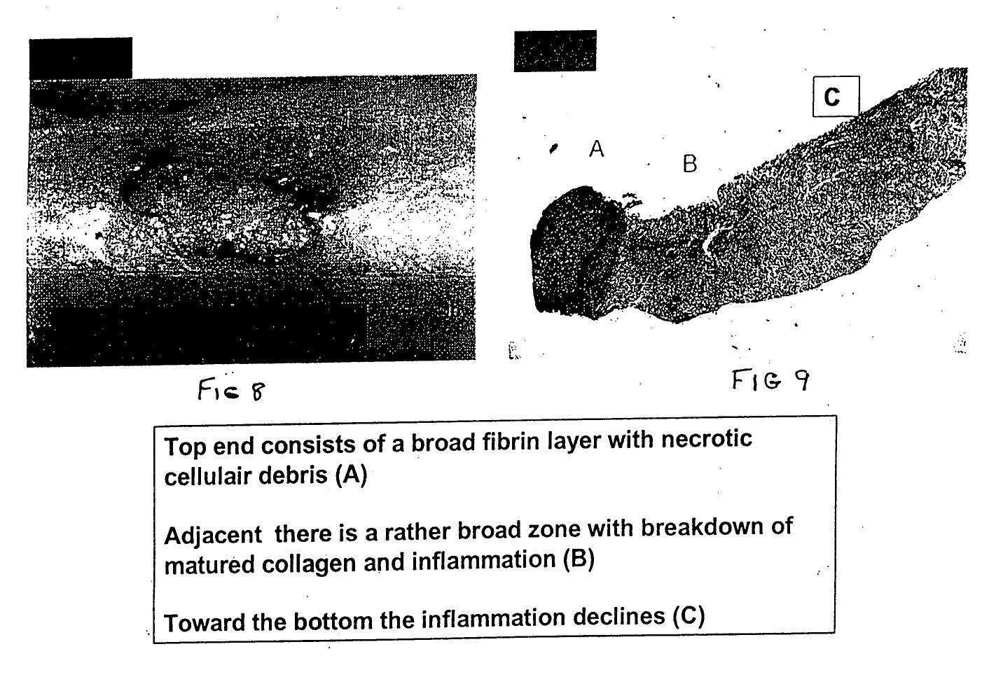

FIG. 6 depicts this wound at the time of commencement of treatment. Prior to entry into the present study. FIG. 7 depicts the healed wound after 30 weeks of treatment. It is noted that after 12 weeks of treatment with the composition, this patient was treated with steroids. This action was noted to delay the healing process and was discontinued. Thus, without the intervention of the steroid treatment, the healing time for this patient would have been shorter.

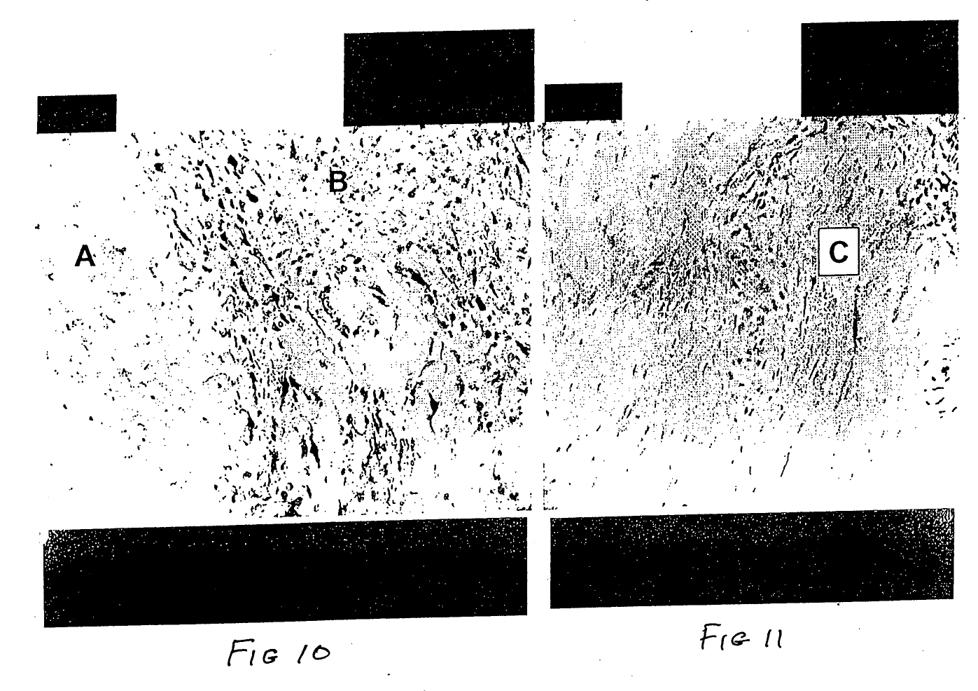

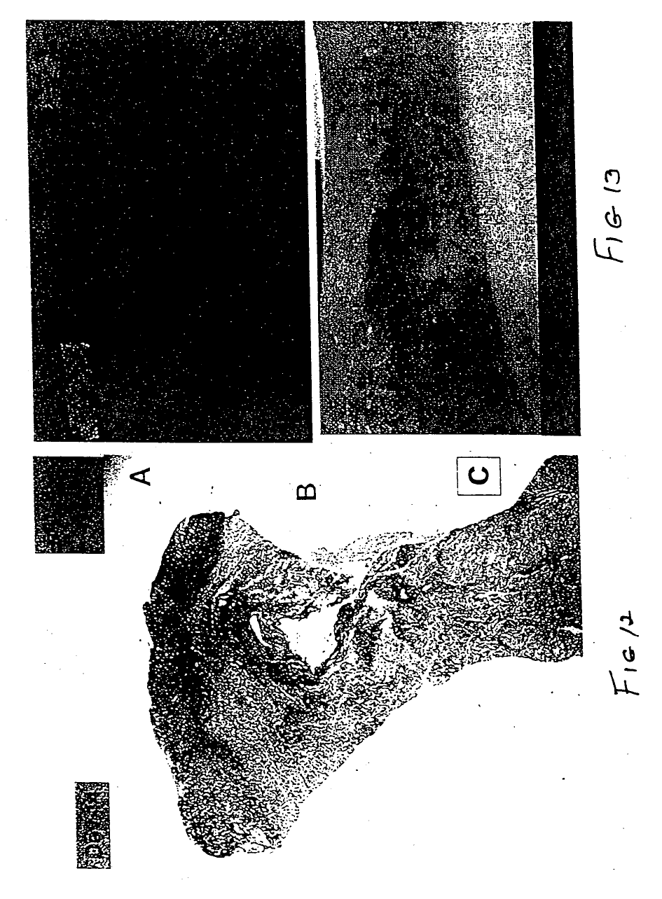

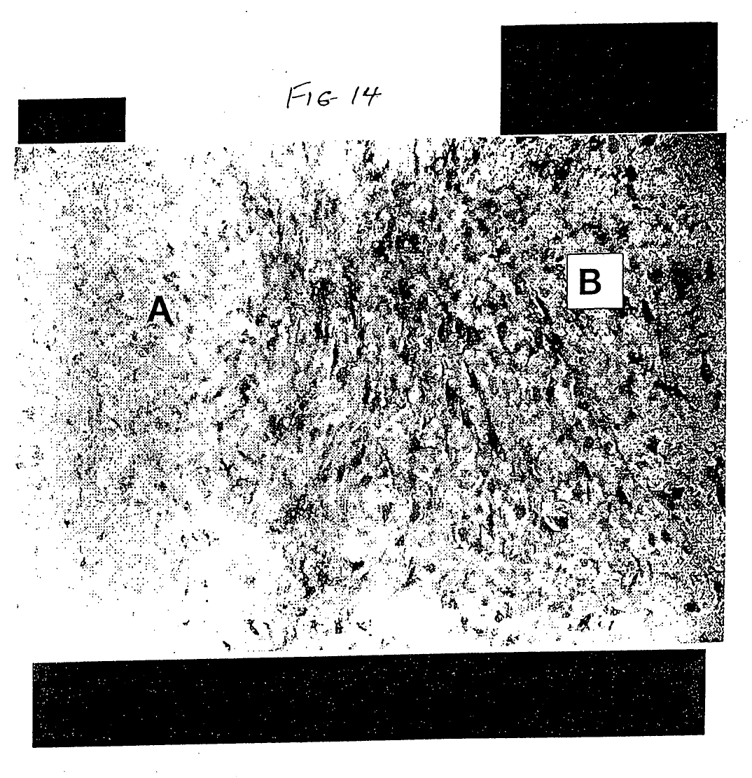

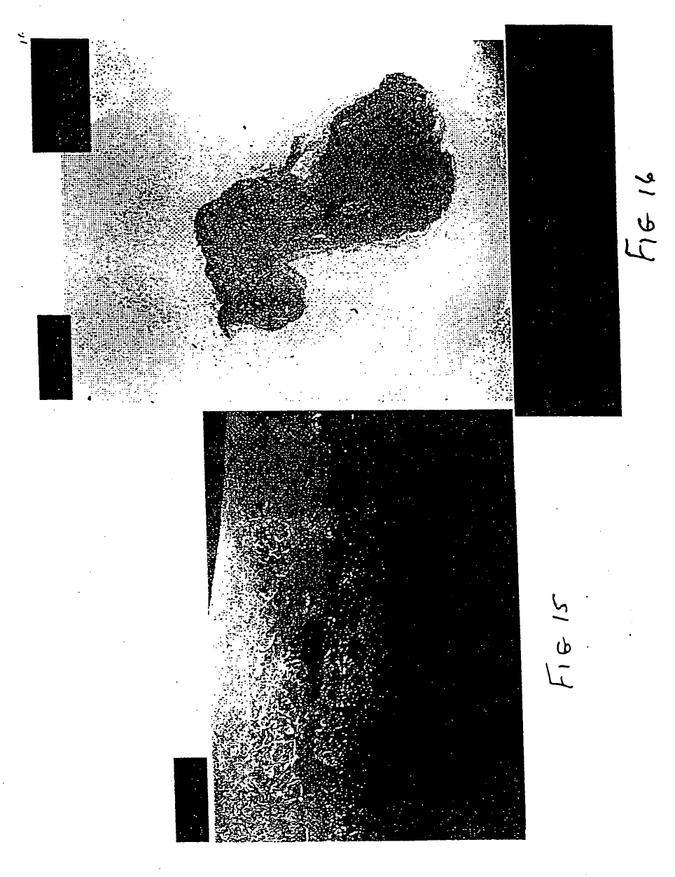

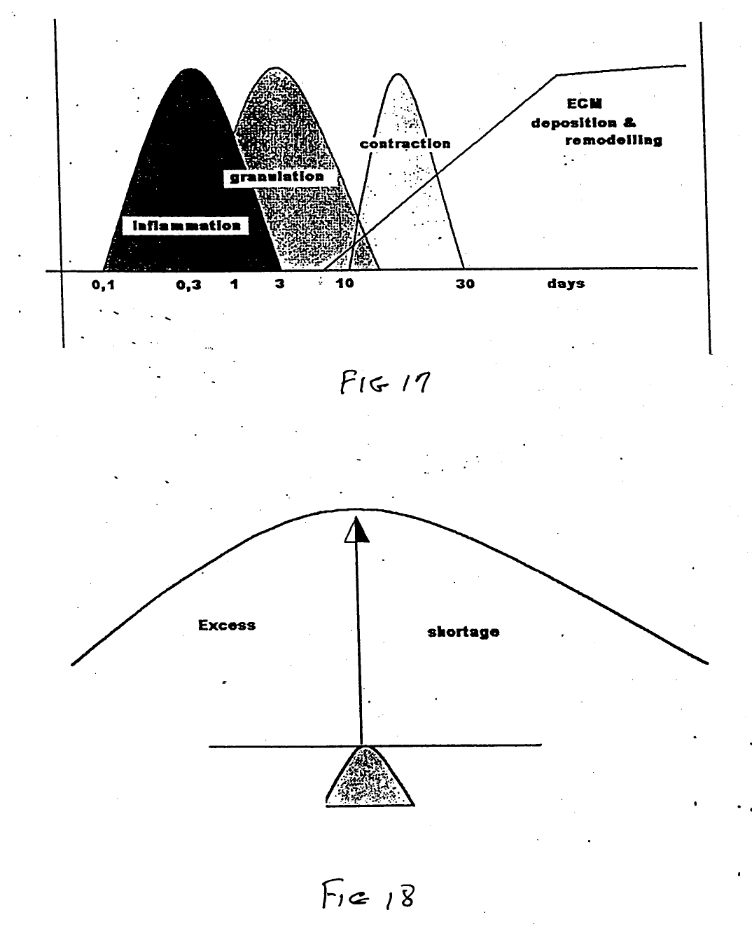

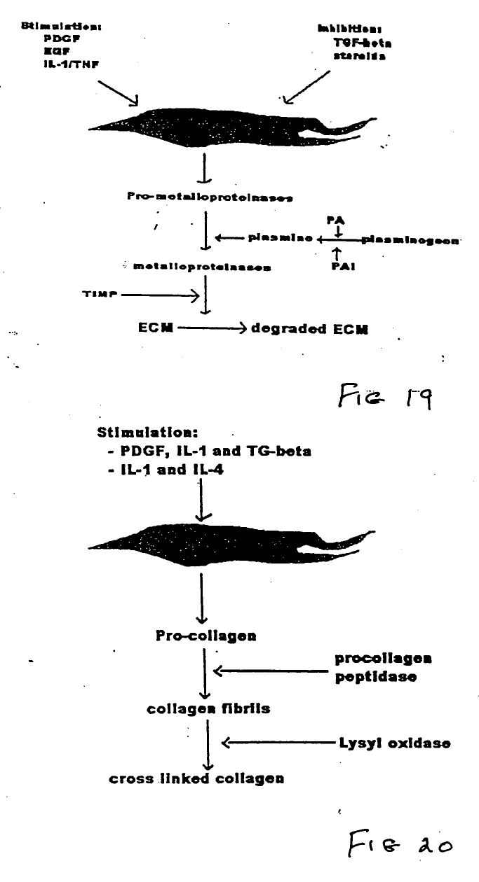

Referring to FIGS. 8 and 9, at Day One, the wound of this patient was about 6 cm long and about 2 cm wide. The wound extended deeply into the leg. A biopsy of the wound is depicted in FIG. 9 wherein a cross-section of the wound is depicted as including Zones A, B and C. Zone A consists of a broad fibrin layer with necrotic cellular debris. Zone B is a rather broad zone with breakdown of matured collagen and inflammation. Zone C is adjacent the bottom of the wound and depicts a decline of inflammation at this location. Examination of the Day One biopsy for MMP-2 prior to the treatment showed fibroblasts in the upper layers of the wound to be expressing high levels of MMP-2 (FIG. 10) This same biopsy depicted no more than a single fibroblast staining positive for MMP-2 in the deeper layers of the wound. As depicted in FIGS. 12 and 13, after 14 days of treatment with the composition, all zones are readily identifiable, with the fibrin cap depicting large accumulations of neutrophils. Zone B at this time of treatment is identifiable directly beneath the fibrin cap and shows less old collagen and the appearing of neo-dermis. FIG. 13 shows the overall appearance of the wound after 14 days treatment and clearly indicates both a “cleaner” wound and reduction in the overall size of the original wound. Biopsies of the wound after 14 days of treatment showed no clear change in the expression of MMP-2 in Zone B (FIG. 14). As shown in FIGS. 15 and 16, after 6 weeks of treatment, the wound was further decreased in size and healing was progressing. A biopsy of the wound at this time showed that the necrotic cap had vanished and the neo-dermis was healthy. Further, the biopsy the expression of MMP-2 within the wound had declined to near zero, coinciding with the healthy appearance of the neo-dermis.

Between the 6^{th} and 12^{th} weeks of treatment of the present patient, steroid treatment was conducted. At week 12, a biopsy of the wound clearly showed that the fibroblasts began again to express MMP-2. Treatment of the wound using steroids was ceased and the wound fully healed within a total treatment time of 30 weeks as shown in FIG. 7.

In one embodiment, the composition of the present invention includes a formulation comprising at least one of zinc ions, rubidium ions, potassium ions, and calcium ions.

Solutions including various of the above-listed ingredients were prepared as follows:

Composition I

| | | |
|---|---|---|
| potassium citrate | 0.895 | moles/l |
| rubidium chloride | 3.1 | millimoles/l |
| zinc chloride | 64 | micromoles/l |

| | |
|---|---|
| citric acid | (sufficient to adjust the pH |
| | of the solution to 5.5) |

Composition II

| | | |
|---|---|---|
| potassium citrate | 0.895 | moles/l |
| rubidium chloride | 3.1 | millimoles/l |
| zinc chloride | 64 | micromoles/l |
| calcium chloride | 0.2 | millimoles/l |

| | |
|---|---|
| citric acid | (sufficient to adjust the pH |
| | of the solution to 5.5) |

Composition III

| | | |
|---|---|---|
| potassium hydroxide | 0.895 | moles/l |
| rubidium chloride | 3.1 | millimoles/l |
| zinc chloride | 64 | micromoles/l |

| | |
|---|---|
| citric acid | (sufficient to adjust the pH |
| | of the solution to 5.5) |

Composition IV

| | | |
|---|---|---|
| potassium hydroxide | 0.895 | moles/l |
| rubidium chloride | 3.1 | millimoles/l |
| zinc chloride | 64 | micromoles/l |
| calcium chloride | 0.2 | millimoles/l |

| | |
|---|---|
| citric acid | (sufficient to adjust the pH |
| | of the solution to 5.5) |

Composition I was employed in Example I above.

Preferably, pharmaceutical grade ingredients are employed in each composition of the present invention.

Compositions I and III were subjected to chemiluminescence assay (indicative of inhibition of production of reactive oxygen species, complement assay (classical pathway, indicative of complement activity). These compositions of the present invention exhibited IC-50 values as follows:

TABLE A

| | Chemiluminescence | Complement |
|---|---|---|
| | Assay | Assay |
| Example I | 10 μl/ml | 9 μl/ml |
| Example II | 36 μl/ml | 28 μl/ml |

Composition II which included potassium hydroxide required a greater amount of citric acid to produce a pH of 5.0, indicating that the potassium citrate employed in Example I was more active, hence the lower IC-50 values exhibited by Composition I. In any event the complement assay results clearly show the effectiveness of the present composition in the modulation of MMPs found in chronic wounds such as diabetic ulcers, decubitus ulcers, and other wounds.

In one embodiment, the composition of the present invention may be incorporated into a pharmaceutically acceptable carrier such as WHITFIELD'S ointment or other suitable creme.

In the aforesaid embodiment, the composition of the present invention, preferably in its creme-type carrier, may be applied directly to an open wound or the like or through the use of a gauze type bandage to which the composition is applied. As desired, the carrier may comprise hydrogels, alginates, aerosol or like carriers depending in part upon the location of the wound or injury or other factors affecting the effective delivery of the composition to the wound or injury.

A preferred composition for use in the treatment of various open wounds comprises 0.895 moles/l potassium citrate, 3.1 millimoles rubidium chloride, 0.2 millimoles/l calcium chloride and 64 micromoles zinc chloride in a solution employing distilled water. The solution is acidified to pH 5.0 employing citric acid.

The preferred composition of the present invention may be modified by eliminating calcium ions, but with some reduction in the efficacy of the composition in treating at least certain wounds. As noted, substitution of potassium hydroxide for potassium citrate in the present composition is permissible, but not preferred, due to the increased need for acid to adjust the pH of the solution to 5.0 and indications are that potassium citrate is more effective than potassium hydroxide. Though present in a relatively small amount, the presence of zinc ions in the solution appear to be important to the desired level of effectiveness of the present composition. This same factor appears true for rubidium ions. Whereas the sources of the inorganic ions of the present composition are given herein, it is to be recognized that other sources of these ions may be acceptable for given applications of the composition. Initial tests have indicated that the quantity of the several inorganic ions in the composition may be varied from the preferred composition without destruction of, but with possible reduction of, the therapeutical efficacy of the composition. In all instances, preferably, the pH of the solution is adjusted to substantially 5.0 thereby imparting-desirable buffering properties to the composition.

In any event, the active ingredients of the present composition have been found to include zinc, potassium, rubidium and/or calcium. Calcium does not appear to be critical to the desired healing process, it does not appear to be detrimental when included in the present composition, and in certain instances is considered desirable. On the other hand, zinc appears to be essential to the healing qualities of the present composition, and rubidium is also strongly indicated for those compositions employed in cancer, ulcer and others of those maladies for which the present compositions have been found useful as healing agents.

Citric acid, preferably, when included in the present composition for pH control purposes has been found effective in such role and its salt (e.g. potassium citrate) appears to provide even greater enhanced therapeutical value to the composition. Other acids for normalizing the pH of present solution, for example hydrochloric acid, may be employed, but are less desirable.

Polyethylene glycol has been found particularly effective as a component of the present solution, in part due to its oxygen scavenging properties.

In one embodiment of the present invention, a channeling agent, such as monoxidil, has been found to be effective in lieu of the potassium ions.

Whereas the compositions of the present invention may include other inactive or relatively inactive ingredients which are biologically relatively inert or inactive, the present inventors have found that at least one or more of the ions of zinc, potassium, rubidium and calcium (in certain compositions) are essential to obtaining the aforenoted dramatic results of wound healing.

During wound repair, for example, different MMPs are produced by multiple cell types. MMP-2 is produced only by inflammatory cells. MMP-9 is produced by keratinocytes as well as inflammatory cells. MMP-2 and MMP-9 act on cleaved collagen better than other MMPs. MMPs are not actively expressed in uninjured skin either in the epidermis or dermis. The idea exists that MMPs are stored in the matrix awaiting activation by migrating cells. Inflamed tissues in chronic wounds exhibit excessively high MMP levels in comparison to normal healing wounds, the excess being in the range of 30% greater MMP levels in chronic wounds.

In accordance with one aspect of the present invention, the compositions of the present invention exhibit those properties which are known-to increase tissue regeneration of chronic open wounds, providing full wound closure of demonstrated non-responding or slow-healing wounds.

At a first level, compositions of the present invention clearly modulate the expression of one or more MMPs, particularly MMP-2 and MMP-9, thereby reducing the levels of these MMPs. At second and further levels, compositions of the present invention function to scavenge oxygen radicals from wound sites, normalizing the pH levels within a wound and thereby developing an environment within the wound which is favorable to healing, possibly rendering the site more amenable to the action of modulation of the MMPs. Still further, the compositions also can reduce inflammation, scavenge free oxygen radicals, reduce scar tissue, and act as a powerful antimicrobal.

Dermal wound healing is recognized as a complex, but orderly process which takes place in injured tissue. Subsequently the injured tissue respond with inflammation, granulation tissue formation, extracellular matrix (ECM) deposition, contraction and remodeling of the deposited collagen. This process is depicted in FIG. 37. The present inventors have found that remodeling results when there is a balance between ECM-synthesis and ECM-degradation. Many different circumstances can influence these processes thus shifting the balance toward a state of excess or shortage of ECM, thereby inhibiting the remodeling process (See FIG. 38). As seen in FIG. 39, fibroblast synthesis of collagen, the major constituent of the dermal tissue, is stimulated by growth factors and cytokines. Soluble pro-collagen peptides are released in the environment of the fibroblasts. Procollagen peptidase cleaves of the terminal peptide chains allow true collagen fibrils to form. Lysyl-oxidase promotes the cross-linking of these fibrils rendering structural stability to the matrix. In the ECM, several types of collagen can be recognized, along with other substances which contribute to the ECM.

The production of MMPs, enzymes that serve to degrade collagen, are also under the influence of growth factors. Stimulating and inhibition factors result in the release of pro-metalloproteinases. These pro-forms are activated by plasmine. Activated matrix metalloproteinases are quickly deactivated by Tissue Inhibitors of metalloproteinases (TIMPs) so that the spatial action of the proteolytic enzyme is limited. The main action of the MMPs is to degrade the collagen. It has to be borne in mind that this scheme is likely to be an oversimplification of what is happening in vivo. For example, (a) plasmine release from plasminogeen is regulated by the action of plasminogeen activator (PA) and plasminogeen Activator Inhibitor (PAI) both of which are also produced by fibroblasts under the influence of growth factors and cytokines; (b) Metalloproteinases can also be activated by other substances as HOCL- from the oxidative burst of granulocytes (H₂O₂+MPO+Cl⁻→HOCl— which is strongly anti-bacterial); (c) metalloproteinases can also be activated by other than TIMP, for instance alpha2-Macroglobulin (anti-protease in serum); and/or (d) metalloproteinases can cleave other molecules than collagen for instance other ECM molecules by cleavage capacity can perhaps also lead to activation of the complement system.

Very little appears to be known about the distribution of MMPs in time. It is known that normal skin shows basic levels of MMP-2, but shows no MMP-9 expression. The present inventors have shown elevated levels of MMPs in chronic wounds.

Irrespective of the complexity of the wound healing mechanism, the present inventors have discovered a combination of metal ions which in solution, preferably substantially at a pH of 5.0, when applied over time, dramatically modulates MMPs. The composition of the present invention is further indicated in the treatment of cancers, psoriasis, and a variety of skin infections, burns, and/or lesions.

1-25. (canceled)

26. A method for therapeutic modulation of one or more matrix metalloproteinases (MMPs), comprising the steps of:
identifying an elevated level of one or more MMPs expressed in tissue;
administering a composition to the tissue to modulate the expression of one or more MMPs, wherein said composition comprises a pharmaceutically effective amount of a combination of zinc and rubidium ions in a physiologically inert carrier;
monitoring the demodulation of the one or more MMPs in the tissue; and
re-administering the composition to the tissue until the one or more MMP levels return near zero.

27. The method of claim 26, wherein the tissue being therapeutically modulated for MMP expression is a chronic wound.

28. The method of claim 27 wherein the chronic wound being treated is an open, full thickness tissue wound.

29. The method of claim 27 wherein the chronic wound being treated is subsurface traumatized tissue.

30. The method of claim 26, wherein the identifying step further comprises taking and examining a biopsy of the tissue to determine whether an elevated level of the one or more MMPs is being expressed.

31. The method of claim 30, wherein the monitoring step further comprises taking and examining a biopsy of the tissue to determine whether the expression of the one or more MMPs has declined.

32. The method of claim 31, wherein the administering and re-administering steps further comprise substantially and fully filling a wound cavity with the composition to ensure that the composition reaches both the outer edges of the wound cavity and the deep, inner recesses of the wound cavity.

33. The method of claim 26, wherein the composition applied in the administering step further comprises a pharmaceutically effective amount of calcium ions.

34. The method of claim 26, wherein the composition applied in the administering step further comprises a pharmaceutically effective amount of potassium ions.

35. The method of claim 26 wherein the composition applied in the administering step further comprises a sufficient amount of an acid to adjust the pH to about 5.0

36. The method of claim 35 wherein the composition applied in the administering step further comprises a sufficient amount of citric acid to adjust the pH to about 5.0.

37. A method for therapeutic modulation of a matrix metalloproteinase (MMP), comprising the steps of:
identifying an elevated level of MMP-2 or MMP-9 expressed in tissue;
administering a composition to the tissue to modulate the expression of MMP-2 or MMP-9, wherein said composition comprises a pharmaceutically effective amount of a combination of zinc and rubidium ions in a physiologically inert carrier;
monitoring the demodulation of the MMP-2 or MMP-9 in the tissue; and
re-administering the composition to the tissue until MMP levels return near zero.

38. The method of claim 37, wherein the tissue being therapeutically modulated for MMP expression is a chronic wound.

39. The method of claim 37, wherein the identifying step further comprises taking and examining a biopsy of the tissue to determine whether an elevated level of MMP-2 or MMP-9 is being expressed.

40. The method of claim 39, wherein the monitoring step further comprises taking and examining a biopsy of the tissue to determine whether the expression of MMP-2 or MMP-9 has declined.
